(19) United States
(12) Patent Application Publication
(10) Pub. No.: US 2013/0096193 A1
(43) Pub. Date: Apr. 18, 2013

(54) CREATINE BETA-ALANINATE: A NOVEL SALT FOR INCREASING ATHLETIC PERFORMANCE

(71) Applicant: Bruce W. Kneller, Howell, NJ (US)

(72) Inventor: Bruce W. Kneller, Howell, NJ (US)

(21) Appl. No.: 13/691,172

(22) Filed: Nov. 30, 2012

Related U.S. Application Data
(63) Continuation of application No. 12/811,623, filed on Jul. 2, 2010, now abandoned.

(51) Int. Cl. C07C 279/14

(52) U.S. Cl. CPC C07C 279/14
USPC 514/547; 560/171

(57) ABSTRACT

Disclosed are creatine β-alaninate, compositions and formulations containing same, and methods of use therefor.

### RELATED APPLICATIONS

This application is a continuation of U.S. application Ser. No. 12/811,623, filed on Jul. 2, 2010, which is a national stage filing under 35 U.S.C. 371 of International Application PCT/US2009/053604, filed Aug. 12, 2009. International Application PCT/US2009/053604 was published under PCT Article 21(2) in English. The specification of this application is incorporated herein by reference.

### BACKGROUND OF THE INVENTION

Nutritional supplements and approaches for enhancing an athlete's muscle performance and the like (e.g., reducing fatigue, increasing strength, increasing endurance, etc.) have become popular exigencies in various sports and bodybuilding regimes. However, as athletes continually strive for improved muscle performance, there is a continuing need for new, more effective technologies to aid in increasing performance.

Creatine monohydrate is a commonly used nutritional supplement. Creatine monohydrate is soluble in water at a rate of 75 milliliters of water per gram of creatine. Ingestion of creatine monohydrate thus also requires ingestion of large amounts of water. Additionally, in aqueous solutions, creatine converts to creatinine via an irreversible, pH-dependent, non-enzymatic reaction. Aqueous and alkaline solutions contain an equilibrium mixture of creatine and creatinine. In acidic solutions, on the other hand, the formation of creatinine is complete. Creatinine is devoid of the ergogenic beneficial effects of creatine and is typically excreted in the urine. It is therefore desirable to provide, for use in individuals, e.g., animals and humans, forms and derivatives of creatine with improved characteristics such as stability and solubility. Furthermore, it would be advantageous to do so in a manner that provides additional functionality compared to creatine monohydrate alone.

### SUMMARY OF THE INVENTION

The present invention relates to compositions and formulations comprising, consisting essentially of, or consisting of hydrosoluble stable organic salts of creatine and β-alanine, as well as to methods for making said compositions and formulations. Additionally, the invention relates to administration of the compound, compositions or formulations to an animal (e.g., a human) as a means for improving athletic function or cognitive function and/or improving the functional effects or toxicity of creatine and/or β-alanine.

In one embodiment the invention relates to a composition or formulation comprising a creatine β-alaninate alaninate salt. In some embodiments the creatine β-alaninate salt has enhanced solubility in aqueous and organic mediums in comparison with creatine. In some embodiments the creatine β-alaninate salt is from about 2 to about 25 times more soluble than creatine. In other embodiments the creatine β-alaninate salt has increased absorbability and/or tissue bioavailability in humans and animals compared to creatine monohydrate. In some embodiments said salt exhibits reduced paraesthesic effect in comparison with an equivalent amount of β-alanine. In certain embodiments the composition is formulated for oral use.

The invention also relates to a method of increasing athletic performance in an animal comprising administering to the animal a composition comprising an effective amount of creatine β-alaninate. In certain embodiments the effective amount is from about 1.00 mg/kg/day to about 150.00 mg/kg/day. In some embodiments the composition is a nutritional supplement. In some embodiments the composition or formulation comprises creatine β-alaninate in an amount of from about 0.100 g to about 15.00 g, inclusive, or from about 5% to about 100%, inclusive.

In certain embodiments the composition increases the skeletal muscle level of creatine in an animal to whom it is administered, increases the skeletal muscle level of phosphocreatine in the animal, increases the skeletal muscle level of ATP in the animal, and/or buffers plasma H+ levels in the animal during, before, and/or after exercise. In some embodiments the composition further comprises other active ingredients; in other embodiments the composition is administered orally.

In a particular embodiment the creatine β-alaninate salt comprises creatine (CAS Registry No. 57-00-1) and 3-alanine.

The invention also relates to a method of increasing solubility of creatine comprising formulating said creatine as a creatine β-alaninate salt, wherein said salt exhibits increased solubility in comparison with an equivalent amount of creatine monohydrate.

The invention also relates to a method of reducing the paraesthesic effect of β-alanine comprising formulating said β-alanine as a creatine β-alaninate salt, wherein said salt exhibits reduced paraesthesic effect in comparison with an equivalent amount of β-alanine. The invention further relates to a method of reducing the paraesthesic effect of β-alanine administration in a mammal comprising administering to said mammal a composition comprising a creatine β-alaninate salt, wherein said salt exhibits reduced paraesthesic effect in comparison with an equivalent amount of β-alanine. That is, an amount of β-alanine formulated as creatine β-alaninate elicits fewer paraesthesic effects (or paraesthesic effects of less severity) in an animal to whom it is administered in comparison with an equivalent amount of β-alanine administered alone. Accordingly, greater amounts of β-alanine can be administered in the form of creatine β-alaninate without eliciting paraesthesic symptoms (or while eliciting milder paraesthesic symptoms) as compared with β-alanine administered alone.

The invention also relates to a method of increasing tissue bioavailability of creatine in a mammal comprising administering to said mammal a composition comprising a creatine β-alaninate salt, wherein said salt exhibits increased tissue bioavailabily in comparison with an equivalent amount of creatine.

The invention further relates to a method of making creatine β-alaninate comprising reacting creatine and β-alanine in an aqueous solution and extracting the resulting creatine β-alaninate salt with an alcohol.

### BRIEF DESCRIPTION OF THE DRAWINGS

FIG. 1 shows the structure of creatine.

FIG. 2 shows the structure of creatinol O-phosphate.

FIG. 3 shows the structure of β-alanine.

FIG. 4 shows the structure of L-alanine.

FIG. 5 shows the structure of creatine β-alaninate (chemical formula C₄H₁₆N₄O₄).

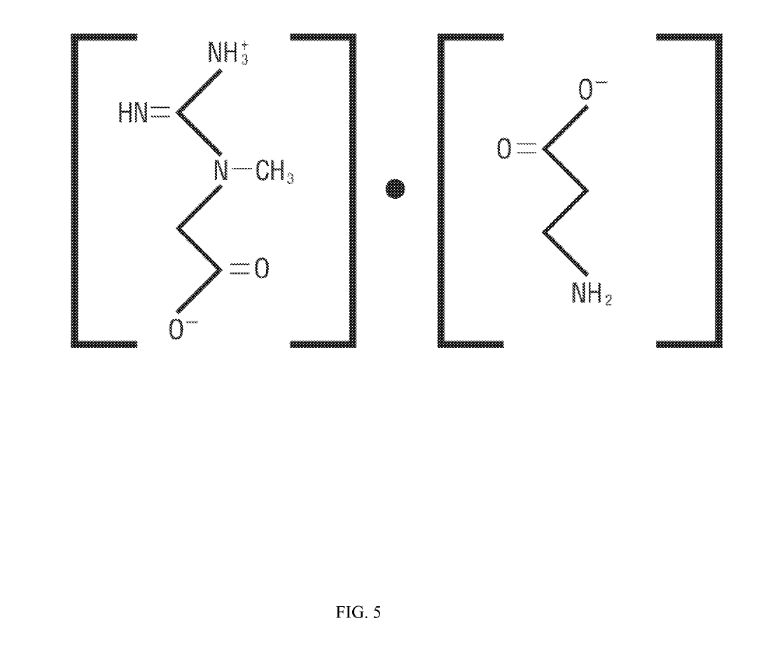

### DETAILED DESCRIPTION OF THE INVENTION

In the following description, for the purposes of explanation, numerous specific details are set forth in order to provide a thorough understanding of the present invention. It will be apparent, however, to one skilled in the art that the present invention is not so limited.

The present invention relates to compositions and formulations comprising, consisting essentially of, or consisting of hydrosoluble stable organic salts of creatine and β-alanine, as well as to methods for making said compositions and formulations. Additionally, the invention relates to administration of the compound, composition or formulation to an animal as a means for improving athletic function or cognitive function.

As used herein, “athletic function” refers to any one or more physical attributes which can be dependent to any degree on skeletal muscle contraction. For example, athletic functions include, but are not limited to, maximal muscular strength, muscular endurance, running speed and endurance, swimming speed and endurance, throwing power, lifting and pulling power. As used herein, “cognitive function” refers to any mental component of brain function. For example, cognitive functions include, but are not limited to, attention, concentration, memory and focus.

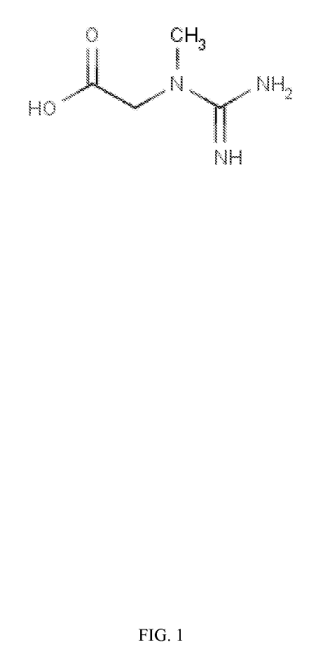

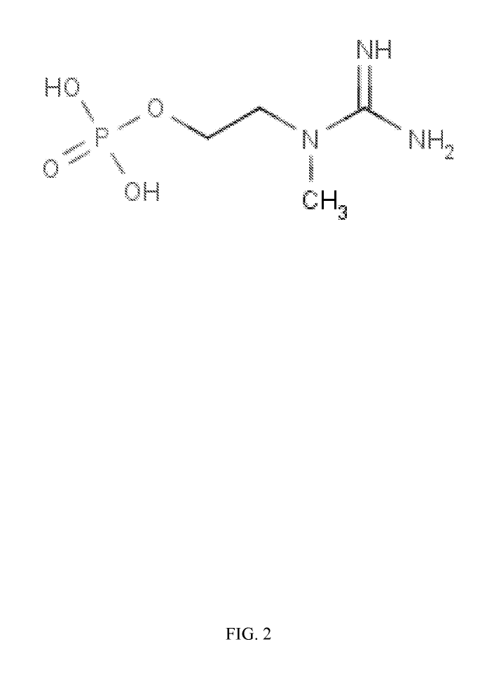

As used herein, “creatine” refers to the chemical N-methyl-N-guanyl Glycine, (CAS Registry No. 57-00-1), also known as (alpha-methyl guanido)acetic acid, N-(aminoiminomethyl)-N-glycine, Methylglycocyamine, Methylguanidoacetic Acid, or N-Methyl-N-guanylglycine, molecular formula of C₄H₉N₃O₂ and a molecular weight of about 131.134 (FIG. 1). Additionally, as used herein, “creatine” also includes glycocyamine (CAS#352-97-6), guanidinopropionic acid (CAS#353-09-3), creatinol (CAS#6903-79-3; FIG. 2), and cyclocreatine (CAS#35404-50-3), as well as any salt, ester, ether, amide, azide, oxide, or chelate thereof or of creatine.

Creatine is a naturally occurring amino acid derived from the amino acids glycine, arginine, and methionine. Although it is found in meat and fish, it is also synthesized by humans. Creatine is predominantly used as a fuel source in muscle. About 65% of creatine is stored in muscle as phosphocreatine (a creatine ion bound to a phosphate ion). Muscular contractions are fueled by the dephosphorylation of adenosine triphosphate (ATP) to produce adenosine diphosphate (ADP); without a mechanism to replenish ATP stores, the human body's supply of ATP would be totally consumed in 1-2 seconds. Phosphocreatine serves as a major source of phosphate from which ADP is regenerated to ATP. Creatine supplementation has been shown to increase the concentration of creatine in the muscle (Clin Sci (Lond). 83(3):367-74), and this supplementation enables an increase in the resynthesis of phosphocreatine (Am J Physiol. 266(5 Pt 1):E725-30), leading to a rapid replenishment of ATP within the first two minutes following the commencement of exercise. It may be through this mechanism that creatine can improve strength and reduce fatigue (Clin Sci (Lond). 84(5):565-71). Furthermore, there is evidence that creatine may have antioxidant properties that may additionally aid post-exercise muscle recovery and recovery from neuronal insults (Free Radio Biol Med. 2006 March 1:40(5):837-49). Thus, creatine supplementation may result in positive physiologic effects on skeletal muscle, including performance improvements during brief high-intensity anaerobic exercise, increased strength, and ameliorated body composition in physically active subjects.

Creatine also mediates remarkable neuroprotection in experimental models of amyotrophic lateral sclerosis, Huntington's disease, Parkinson's disease, and traumatic brain injury. Also, oral creatine administration to experimental animals has been shown to result in a remarkable reduction in ischemic brain infarction and neuroprotection after cerebral ischemia (J Neurosci. 2004 Jun. 30; 24(26):5909-12). Hydrosoluble creatine monohydrate salts are obtainable and have been described elsewhere (see, for instance, U.S. Pat. No. 5,973,199, incorporated herein in its entirety by reference).

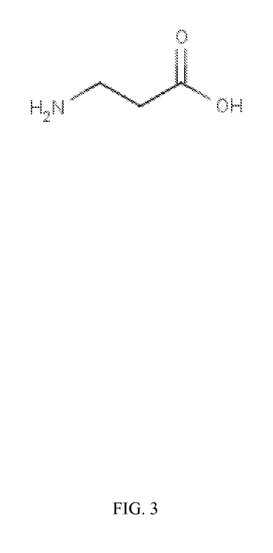

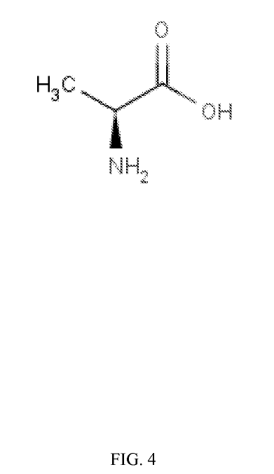

β-alanine (CAS Registry No. 107-95-9, sometimes known as 2-Carboxyethylamine or 3-Aminopropanoic acid), having a molecular formula of C₃H₇NO₂ and a molecular weight of about 89.0933, is a naturally occurring beta amino acid. Beta amino acids are amino acids in which the amino group is at the β-position from the carboxylate group (i.e., two atoms away, FIG. 3). Unlike its normal counterpart, L-α-alanine (FIG. 4), β-alanine has no chiral center. β-alanine is not used in the biosynthesis of any major proteins or enzymes. It is formed in vivo by the degradation of dihydrouracil and carnosine. It is a component of the naturally occurring peptides carnosine and anserine and also of pantothenic acid (vitamin B5), which itself is a component of coenzyme A. Under normal conditions, β-alanine is metabolized into acetic acid. β-alanine is the rate-limiting precursor of carnosine. Supplementation with β-alanine has been shown to increase the concentration of carnosine in muscles, decrease fatigue in athletes, and increase total muscular work done (J Appl Physiol 103: 1736-1743; Amino Acids 32(2):225-33). It is well known by those skilled in the art that increasing muscle carnosine increases lactic acid H⁺ ion buffering capacity and lactic acid H⁺ ion accumulation is the major and fundamental cause of rapid fatigue.

β-Alanine supplementation may increase athletic performance by reducing fatigue or reducing the time to the onset of fatigue associated with lactic acid/H⁺ accumulation during exercise or athletic activity. Typically studies have used β-alanine supplementation strategies of multiple doses of 400 mg or 800 mg, administered at regular intervals for up to eight hours, over periods ranging from 4 to 10 weeks (Amino Acids 32 (2):225-33; Amino Acids 30:279-289). After a 10 week supplementation strategy, the reported increase in intramuscular carnosine content was an average of 80.1% (range 18 to 205%) (Amino Acids 32(2):225-33).

L-histidine, with a pKa of 6.1, is a relatively weak buffer over the physiological intramuscular pH range; however, when bound to other amino acids this increases nearer to 6.8-7.0. In particular, when bound to β-alanine the pKa value is 6.83 (Journal of Physiology 92:336-343), making this a very efficient intramuscular buffer. Furthermore, because of the position of the beta amino group, β-alanine dipeptides are not incorporated into proteins and thus can be stored at relatively high concentrations (millimolar). Occurring at 17-25 mmol/kg (dry muscle) (Eur. J. Appl. Physiol. 64:47-50), carnosine (β-alanyl-L-histidine) is an important intramuscular buffer, constituting 10-20% of the total buffering capacity in type I and II muscle fibers.

β-Alanine provided in solution or as powder in gelatin capsules, however, causes paraesthesia when ingested in amounts above 10 mg per kg body weight (Amino Acids 30:279-289). This is variable among individuals; mild symptoms may be experienced by some individuals even at 10 mg per kg body weight, significant symptoms in a majority of individuals at 20 mg per kg bodyweight, and severe symptoms at 40 mg per kg body weight (Amino Acids 30:279-289). Typically, the paraesthesia begins about 20 minutes after the oral administration of β-alanine and may last for up to 1 hour. However, an equivalent amount (equimolar) to 40 mg per kg body weight, ingested in the form of histidine-containing dipeptides in chicken broth extract, did not cause paraesthesia (Amino Acids 30:279-289).

It is probable that the paraesthesia, a form of neuropathic pain that may be described as a sensation of tingling, pricking, or numbness of a person's skin with no apparent long-term physical effect, is transient, resulting from high peak blood-plasma concentrations of β-alanine. Greater quantities, ingested in the form of β-alanine and histidine dipeptides or β-alanine and methylhistidine dipeptides (e.g., carnosine and anserine) in meat, do not cause the same symptoms. In these circumstances the β-alanine absorption profile is flattened but sustained for a longer period of time (Amino Acids 30:279-289).

The creatine β-alaninate compositions and formulations of the invention harness both the muscle-enhancing and neuroprotective effects of creatine with the fatigue-decreasing and muscle enhancing activity afforded by β-alanine. The novel organic compound can be used in sports nutrition as an ergogenic aid to increase strength, muscle volume and/or size, while affording improved capacity of concentration and mental focus during physical exertion.

During the course of work described herein, it was surprisingly observed that the ionic salt creatine β-alaninate allowed for significantly higher net oral dosing of β-alanine before the onset of paresthesia was observed. In these experiments, single doses of up to 55.00 mg/kg of creatine β-alaninate were administered orally in gelatin capsules to human subjects (the equivalent of administering about 22.25 mg/kg of β-alanine since the ionic salt creatine β-alaninate mass consists of about 40.45% β-alanine) before the onset of parasthesia was noted. That is, in these experiments, roughly 100% more β-alanine could be administered as creatine β-alaninate before eliciting symptoms of paraesthesia as compared with β-alanine alone. To the best of the inventor's knowledge, no other ionic salts of β-alanine exhibit this benefit, thus making creatine β-alaninate unique in this regard. Without wishing to be bound by theory, it may be that the ionic salt formed between creatine and β-alanine, while chemically different from dipeptide salts of β-alanine such as anserine or carnosine, also results in a flattened and sustained absorption curve similar to β-alanine-containing dipeptides. The inventor believes that the oral ingestion of encapsulated creatine β-alaninate yields a peak concentration (Cmax) less than equimolar amounts of orally ingested β-alanine but greater than that of orally ingested β-alanine-containing dipeptides such as anserine or carnosine. The inventor also believes that the time to peak concentration (Tmax) of orally ingested, encapsulated creatine β-alaninate is longer than that of equimolar amounts of orally ingested β-alanine but less than that of orally ingested β-alanine-containing dipeptides such as anserine or carnosine.

Creatine β-alaninate is a white to off-white, crystalline solid compound that is hygroscopic and 2 to 25 times more soluble in water than creatine monohydrate. Ingestion of creatine β-alaninate promotes strength, endurance, recovery, and lean tissue and decreases fat tissue due to the creatine or creatine-like cation content, as well as providing a beneficial, physiological lactic acid/H⁺ buffering effect due to the β-alanine anion content. Creatine β-alaninate may be administered to subjects (e.g., humans) with or without a high protein diet (about 1.25 to 2.0 grams protein/kilogram of body mass) and proper anaerobic training program in order to increase the variables associated with athletic function for the purpose of enhancing athletic performance. The oral, daily dose of a creatine β-alaninate can be from about 0.100 grams to about 15.00 grams per day; a preferred daily dosing schedule is a dose of about 1.50-3.00 grams administered 30-90 minutes before exercise or athletic activity in order to achieve optimal absorption and adequate muscle cell concentration, with a second dose of about 1.50-3.00 grams administered 30-90 minutes after the cessation of exercise or athletic activity. As used herein, the term “about” refers to a +/−10% variation from the nominal value. It is to be understood that such a variation is always included in any given value provided herein, whether or not it is specifically referred to.

Creatine β-alaninate can be made by any suitable method. Exemplary (non-limiting) embodiments of methods of making a creatine β-alaninate are described in the Examples below.

Creatine β-alaninate can be administered before, concurrent with, or after other optional components such as other active ingredients. In some embodiments the nutritional supplement composition comprising a creatine β-alaninate contains one or more of the following ingredients, preferably as an active ingredient:

Carbohydrates including, but not limited to, isomaltulose, trehalose, maltodextrin, glucose, sucrose, fructose, lactose, amylose, and/or ribose;

Water soluble vitamins including, but not limited to, Vitamin C, Vitamin B1, Vitamin B2, Vitamin B3, Vitamin B5, Vitamin B6, Vitamin B12, and/or Vitamin K;

Minerals including, but not limited to, calcium, sodium, potassium, chromium, vanadium, magnesium, and/or iron (and derivatives)(preferably in amounts less than the RDA);

Amino acids including, but not limited to L-arginine, L-ornithine, L-glutamine, L-tyrosine, L-taurine, L-leucine, L-isoleucine, and/or L-valine (and derivatives);
Dipeptides and tripeptides including, but not limited to, L-Carnitine, Carnosine, Anserine, Balenine, Kyotorphin, and/or Glutathione (and derivatives);
Nutraceutically acceptable stimulants including, but not limited to, methylxanthines (e.g.—caffeine) and/or glucuronolactone (and derivatives);
Nutraceutically acceptable hypoglycemic agents including, but not limited to, alpha-lipoic acid and its derivatives, cinnamon bark, bitter melon extracts, Gymnema. Sylvestre extracts, 4-hydroxy-isoleucine, corosolic acid, pterostilbene and/or D-pinitol (and derivatives);
Creatine and its salts (e.g., creatine monohydrate), esters (e.g., creatine ethyl ester), chelates, amides, ethers (and derivatives);
glycocyamine, guanidinopropionic acid, creatinol, and cyclocreatine;
Adenosine triphosphates and its disodium salt;
Glycerol and glycerol monostearate;
Mannitol;
Sorbitol; and
Dextrin.

Preferably the composition or formulation comprises from about 5% to about 100% (by weight) creatine β-alaninate, more preferably about 20% to about 100% creatine β-alaninate, and even more preferably about 50% to about 100% creatine β-alaninate.

As used herein, the terms “nutraceutical” and nutraceutically acceptable” are used herein to refer to any substance that is a food or part of a food and provides medical or health benefits, including the prevention and treatment of disease. Hence, compositions falling under the label “nutraceutical” or “nutraceutically acceptable” may range from isolated nutrients, nutritional or dietary supplements, and specific diets to genetically engineered designer foods, herbal products, and processed foods such as cereals, soups, and beverages. In a more technical sense, the term has been used to refer to a product isolated or purified from foods, and generally sold in medicinal forms not usually associated with foods and demonstrated to have a physiological benefit or provide protection against chronic disease.

As used herein, the term “derivative” can include salts, esters, ethers, amides, chelates, lactone forms, hydrates, or complexes of stated chemicals. Such derivatives can also include stereoisomers or structural isomers, so long as the derivative operates similarly and produces the desired effect. Alternatively, the derivative can be a precursor to the stated chemical, which subsequently undergoes a reaction in vivo to yield the stated chemical or derivative thereof. By way of non-limiting example only, ubiquinol is a useful derivative of ubiquinone, and acetyl-L-carnitine is a useful derivative of L-carnitine, ketoisocaproic acid is a useful derivative of L-leucine, and R-dihydrolipoic acid is a useful derivative of R-α-lipoic acid.

The compositions and formulations of the invention may contain pharmaceutically, e.g., nutraceutically, acceptable excipients, according to methods and procedures well known in the art. As used herein, “excipient” refers to substances that are typically of little or no therapeutic value, but are useful in the manufacture and compounding of various pharmaceutical preparations and which generally form the medium of the composition. These substances include, but are not limited to, coloring, flavoring, and diluting agents; emulsifying, dispersing and suspending agents, ointments, bases, pharmaceutical solvents; antioxidants and preservatives; and miscellaneous agents. Suitable excipients are described, for example, in Remington's Pharmaceutical Sciences, which is incorporated herein by reference in its entirety.

The compositions and formulations according to the present invention can further comprise one or more acceptable carriers. A wide number of acceptable carriers are known in the nutritional supplement arts, and the carrier can be any suitable carrier. The carrier need only be suitable for administration to animals, including humans, and be able to act as a carrier without substantially affecting the desired activity of the composition. Also, the carrier(s) may be selected based upon the desired administration route and dosage form of the composition. For example, the nutritional supplement compositions according to the present invention are suitable for use in a variety of dosage forms, such as liquid form and solid form (e.g., a chewable bar or wafer). In desirable embodiments, as discussed below, the nutritional supplement compositions comprise a solid dosage form, such as a tablet or capsule. Examples of suitable carriers for use in tablet and capsule compositions include, but are not limited to, organic and inorganic inert carrier materials such as gelatin, starch, magnesium stearate, talc, gums, silicon dioxide, stearic acid, cellulose, and the like. Desirably, the carrier is substantially inert, but it should be noted that the nutritional supplement compositions of the present invention may contain further active ingredients in addition to creatine β-alaninate.

Creatine β-alaninate may be prepared via several syntheses. One method of preparation is via reacting equimolar amounts of creatine and β-alanine in aqueous or hydroalcoholic concentrated solution or in a water-immiscible solvent (or mixture of solvents), at temperatures ranging from room temperature to 50° C. Additionally, the reaction may be induced to proceed through the melting of β-alanine (melting point of about 200° C.), forming a liquid reaction medium, and adding creatine, followed by a subsequent extraction of the salt from the reaction mixture with cyclohexane.

According to a preferred embodiment, the creatine β-alaninate salt can be prepared by reacting creatine with an equimolar amount of β-alanine in ethyl acetate (or in a mixture of equal parts ethyl acetate and ethanol) until complete formation of the salt. The solution can be optionally concentrated and, upon cooling, the crystallized salts may be filtered and washed with ethyl acetate (or a mixture of ethyl acetate and ethanol). Alternatively, the procedure can be carried on by reacting excess β-alanine with creatine in ethyl acetate (or a mixture of ethyl acetate and ethanol).

Advantageously, creatine β-alaninate can be used as a composition, either alone or as part of a more complex composition containing any number of additional ingredients. It will be apparent to those skilled in the art which specific ingredients may be beneficially included in such compositions.

Furthermore, creatine β-alaninate, as a compositional ingredient, may be administered in any form common in the art. For example, the compositional ingredient may be administered in the form of a powder to be mixed in liquid or in a solid dosage form such as a tablet, capsule or caplet. Additionally, creatine β-alaninate may be suspended or dissolved in any pharmaceutically acceptable carrier or vehicle medium for injection. As such, it may be combined with any number of commonly accepted excipients, as is regularly practiced in the art.

The compositions and methods of the present invention may provide significant increase or improvement in athletic performance, e.g., muscle size, and/or muscle strength, and/or muscle endurance in individuals. As used herein, “athletic performance” and/or “athletic functions” refers to one or more physical attributes which can be dependent to any degree on skeletal muscle contraction. For example, athletic performance and/or athletic functions include, but are not limited to, maximal muscle power, muscular endurance, running speed and endurance, swimming speed and endurance, throwing power, lifting and pulling power.

While it is expected that the compositions and methods of the present invention will be of particular importance to bodybuilders and other athletes, the usefulness of compositions and methods of the invention is not limited to those groups. Rather, any individual may beneficially use the compositions and methods of the invention. Indeed, the disclosed compositions and methods have application to all animals, including mammals, birds and reptiles. As used herein, the term “animal” includes all members of the animal kingdom, preferably mammals (e.g., dogs, horses, cows, mules), more preferably humans. For example, the nutritional supplements of the invention may have beneficial effect for competitive animals (e.g., racehorses, show horses, racing dogs (e.g., greyhounds), bird dogs, show dogs) and work animals (e.g., horses, mules and the like) in whom an increase in muscle performance is desirable.

The compositions and formulations according to the present invention may be employed in methods for supplementing the diet of an individual, e.g., an athlete, and/or for enhancing an individual's muscle mass and/or muscle size and/or strength, and/or endurance. Accordingly, the present invention provides methods of supplementing the dietary intake of an individual comprising administering to the individual an effective amount of a composition (e.g., a creatine β-alaninate or a nutritional supplement comprising a creatine β-alaninate) according to the present invention to increase athletic performance or athletic function is said individual. The invention also relates to methods of improving athletic performance and/or athletic function in an individual comprising administering an effective amount of a creatine β-alaninate (alone or in combination with other agents, e.g., in a nutritional supplement) to the individual.

As used herein, an “effective amount” of compositions of the present invention is defined as an amount effective, at dosages and for periods of time necessary, to achieve the desired result. The effective amount of compositions of the invention may vary according to factors such as age, sex, and weight of the individual. Dosage regime may be adjusted to provide the optimum response. Several divided doses may be administered daily, or the dose may be proportionally reduced as indicated by the exigencies of an individual's situation. As will be readily appreciated, a composition in accordance with the present invention may be administered in a single serving or in multiple servings spaced throughout the day. As will be understood by those skilled in the art, servings need not be limited to daily administration, and may be on an every second or third day or other convenient effective basis. The administration on a given day may be in a single serving or in multiple servings spaced throughout the day depending on the exigencies of the situation.

The present invention discloses a hydrosoluble stable organic salt of creatine and β-alanine, i.e., creatine β-alaninate, characterized by high water solubility, i.e., from 2 to 25 times higher than that of creatine itself, and having a melting point about 140° to about 180° C. with a molecular weight range of about 218 to about 224. The present invention describes processes for the preparation of the salt and methods for administering the salt to an animal, such as a human. The present invention also discloses methods of using effective amounts of creatine β-alaninate for the regulation of athletic function in animals.

The invention also relates to a dietary or nutritional supplement or nutraceutical, e.g., a food supplement, a food composition, comprising, consisting essentially of or consisting of a creatine salt, and to methods of making and using said creatine salt. In one embodiment of the invention the creatine salt is creatine β-alaninate, i.e., a stable hydrosoluble salt consisting of (1) a creatine or creatine-like cation and (2) the ionic form of the amino acid, β-alanine as the β-alaninate anion. The creatine β-alaninate of the invention is superior to existing ionic salts of creatine due to its increased solubility, increased oral bioavailability, and ability to produce increased athletic function. Moreover the creatine β-alaninate of the invention is superior to existing β-alanine salts due to the reduction in paraesthesia which is typical with oral ingestion of other β-alanine salts. The compound of the invention can be administered as a component of a nutritional supplement; the nutritional supplement may optionally contain amounts of various other nutrients.

In one embodiment the invention relates to a composition comprising the ionic salt creatine β-alaninate which has increased solubility in organic mediums and aqueous solutions as compared to creatine; has increased tissue bioavailability in animals as compared to creatine; and/or has increased absorbability as compared to creatine. The composition can have one or more (combinations and subcombinations) improved functional properties.

In some embodiments the composition increases the skeletal muscle level of creatine in an animal; increases the skeletal muscle level of phosphocreatine in an animal; increases the skeletal muscle level of ATP in an animal; increases athletic performance in an animal; and/or helps to buffer plasma H⁺ levels in an animal during, before, and/or after exercise. The composition can have one or more (combinations or subcombinations) of the listed physiological effects.

In preferred embodiments, the composition is a nutritional supplement comprising the ionic salt creatine β-alaninate in an amount of from about 0.100 g to about 15.00 g, inclusive. In other embodiments the composition is a nutritional supplement comprising the ionic salt creatine β-alaninate in an amount of from about 5% to about 100%, inclusive. In certain embodiments, the composition comprises additional active ingredients and/or is formulated for oral use.

The invention also relates to a method of increasing athletic performance in an animal comprising administering to the animal a composition comprising the ionic salt creatine β-alaninate. In certain embodiments, the composition is a nutritional supplement comprising the ionic salt creatine β-alaninate in an amount of from about 0.100 g to about 15.00 g, inclusive. In other embodiments, the composition is a nutritional supplement comprising the ionic salt creatine β-alaninate in an amount of from about 5% to about 100%, inclusive.

In some embodiments, the administered composition increases the skeletal muscle level of creatine in said animal; increases the skeletal muscle level of phosphocreatine in said animal; increases the skeletal muscle level of ATP in said animal; and/or buffers plasma H⁺ levels in said animal during, before, and/or after exercise. In some embodiments, the administered composition comprises other active ingredients and/or is administered orally.

The embodiments set forth in the present application are provided only to illustrate various aspects of the invention, and additional embodiments and advantages of the compositions and methods of the present invention will be apparent to those skilled in the art. The teachings of all references cited herein are incorporated herein by reference. The invention will be further exemplified by the following non-limiting examples.

### EXAMPLES

### Examples of Syntheses

### Example 1

### Procedure:

1) About 9.0 g (0.1 mol) of β-alanine (99% purity) is added to 100 ml of ethyl acetate in a beaker. A stir bar was placed inside and the mixture stirred for about 10 min.
2) About 14.9 g (0.1 mol) of creatine monohydrate is added to the stirred suspension at 20-25° Celsius, and the mixture is allowed to stir for about 3 hours at 25° Celsius.
3) A white to off-white, finely crystalline product is obtained and is separated out by filtering. The filtrate is then discarded, and the crystalline residue is collected. The crystalline residue is creatine β-alaninate.

### Example 2

### Procedure:

1) About 9.0 g (0.1 mol) of β-alanine (99% purity) is added to 20 milliliters of distilled/deionized H₂O in a beaker.
2) The mixture from STEP 1 is heated to about 30° Celsius and stirred mechanically for about 15 minutes.
3) 14.9 g (0.1 mol) of creatine monohydrate is added to the mixture from STEP 3 and is allowed to stir for about 30 minutes until concentrated (note that the mixture will be have a slurry-like consistency) and cooled to 5° Celsius.
4) The product mixture from STEP 3 is filtered and then the solid residue is collected.
5) The collected product from STEP 4 is suspended in 50 milliliters of absolute ethanol to remove any residual water.
6) The mixture from STEP 5 is filtered, and the solid crystalline residue is allowed to dry for about 24 hours and then is recovered. This solid, crystalline residual material is creatine β-alaninate.

### Example 3

### Procedure

1. About 14.9 g (0.1 moles) of creatine monohydrate and about 9.0 g (0.1 moles) of β-alanine are dissolved in the minimum amount of water.
2. Isobutanol or butanol is added to the solution from STEP 1, and the mixture thus obtained concentrated under vacuum by means of azeotropic distillation.
3. A residue is obtained from STEP 2 which is taken up with acetone, filtered off and dried under vacuum in a thermostatic oven at 40° Celsius overnight. The resultant residue is creatine β-alaninate.
4. The salt thus obtained from STEP 3 may be crystallized with methanol, resulting in macrocrystals having the same physic-chemical properties as the raw material.

### Examples of Use

The servings set forth in these examples are designed for athlete with a body mass of about 70 kilograms. Daily values can be increased or decreased depending on the body mass of the individual athlete and individual needs and requirements.

### Example 4

In this example, an athlete consumes four servings of a food supplement as described herein daily; that is, a serving of the food supplement about every six hours. Each serving of the food supplement is about 2.00 grams and contains 2.00 grams of creatine β-alaninate. Each 2.00 gram serving is administered as a powder dissolved into about 300-500 milliliters of water or fruit juice to provide a liquid drink.

### Example 5

In this example, an athlete consumes two servings of the food supplement as described herein daily; typically the athlete will consume one serving of the food supplement about 30-90 minutes before exercise or athletic activity and the second serving of the food supplement immediately after the cessation of exercise or athletic activity. Each serving is about 99.00 grams and contains the following:
creatine β-alaninate 3.00 grams; Maltodextrin 50.00 grams; and Whey Protein Concentrate 46.00 grams.

Each approximate 99.00 gram serving is mixed in about 500-1000 milliliters of water or fruit juice to provide a liquid drink.

### Example 6

In this example, an athlete consumes one serving of the food supplement described herein daily; typically the athlete will consume a serving of the food supplement about 60 minutes before exercise. Each serving is about 85.85 grams and contains the following:
Creatine β-alaninate 3.00 grams; Magnesium Creatine Chelate 2.00 grams; L-Leucine 5.00 grams; Propionyl-L-Carnitine 1.00 gram; Ubiquinone 0.100 grams; L-Taurine 3.00 grams; L-Glutamine 7.50 grams; L-Tyrosine 2.00 grams; Disodium ATP 0.200 grams; Partially Hydrolyzed Guar Gum 5.00 grams; Isomalulose 15.00 grams; Trehalose 15.00 grams; Glucose 15.00 grams; Calcium Phosphate 3.00 grams; Calcium Citrate 2.00 grams; Caclium Bicarbonate 5.00 grams; Carnosine 2.00 grams and Potassium R-α-Lipoic Acid 0.050 grams.

Each approximate 85.85 gram serving is mixed in about 500-750 milliliters of water or juice to provide a liquid drink.

What is claimed is:

1. A method of increasing solubility of creatine comprising formulating said creatine as a creatine β-alaninate salt, wherein said salt exhibits increased solubility in comparison with an equivalent amount of creatine monohydrate.

2. A method of reducing the paraesthesic effect of β-alanine comprising formulating said β-alanine as a creatine β-alaninate salt, wherein said salt exhibits reduced paraesthesic effect in comparison with an equivalent amount of β-alanine.

3. A method of reducing the paraesthesic effect of β-alanine administration in a mammal comprising administering to said mammal a composition comprising a creatine β-alaninate salt, wherein said salt exhibits reduced paraesthesic effect in comparison with an equivalent amount of β-alanine.

4. A method of making creatine β-alaninate comprising reacting creatine and β-alanine in an aqueous solution and extracting the resulting creatine β-alaninate salt with an alcohol.
